(19) United States
(12) Patent Application Publication
(10) Pub. No.: US 2026/0283534 A1
(43) Pub. Date: Sep. 24, 2026

(54) MRI-REGISTERED CORTICO-HYDRAULIC DIGITAL TWIN FOR BAYESIAN SUPER-RESOLUTION ESTIMATION OF CSF FLOW, EXTRACELLULAR-SPACE DYNAMICS, NEURAL SOURCE PATTERNS, AND FUNCTIONAL NEUROMODULATION TARGETS USING hdEEG AND FAST-EIT

(71) Applicant: Brain Electrophysiology Laboratory Company, LLC, Eugene, OR (US)

(72) Inventor: Don M. Tucker, Eugene, OR (US)

(21) Appl. No.: 19/693,710

(22) Filed: Jun. 1, 2026

Related U.S. Application Data
(63) Continuation-in-part of application No. 19/072,686, filed on Mar. 6, 2025.
(63) Continuation-in-part of application No. 19/097,519, filed on Apr. 1, 2025, now Pat. No. 12,663,866.
(63) Continuation-in-part of application No. 19/350,041, filed on Oct. 5, 2025.

(51) Int. Cl. A61B 5/372; A61B 5/053; A61B 5/291; A61N 1/02; A61N 1/36

(52) U.S. Cl. CPC A61B 5/372; A61B 5/053; A61B 5/291; A61N 1/025; A61N 1/36031; A61B 2562/06

(57) ABSTRACT

A system and method estimate sleep-dependent intracranial fluid transport using a T1-registered or atlas-scaled probabilistic cerebrospinal-fluid-flow prior atlas calibrated by FAST-EIT or FAST-ECS measurements. The atlas represents high-resolution probabilities of cerebrospinal-fluid spaces, perivascular routes, parenchymal extracellular-space compartments, and outflow pathways, together with driver-specific response maps for cardiac pulsatility, respiration, vasomotor oscillations, sleep stage, slow oscillations, REM phasic activity, arousal, posture, and transcranial stimulation. The atlas is nonlinearly registered to a subject's T1 MRI or scaled to a subject's head model. EEG, impedance, and physiological recordings are acquired during sleep, and an electrical forward model predicts boundary impedance from atlas-derived extracellular-space and conductivity changes. Bayesian super-resolution fits the high-resolution fluid-transport prior to lower-native-resolution FAST-EIT/FAST-ECS data to estimate posterior maps of CSF flow, perivascular exchange, extracellular-space expansion, parenchymal transport, and glymphatic clearance capacity. Multi-night assessments, including four-night WISP or high-density EEG/EIT recordings, may be combined with dried blood or dried plasma spot biomarkers to classify clearance-reserve stage and validate amyloid, tau, glial, neurodegenerative, synucleinopathy, or mixed-pathology risk. Stimulation or sham challenge may be used to compute target engagement and model adequacy.

### RELATED APPLICATIONS

This application is a continuation-in-part of U.S. Ser. No. 19/072,686, filed Mar. 6, 2025, and U.S. Ser. No. 19/097,519, filed Apr. 1, 2025, and U.S. Ser. No. 19/350,041, filed Oct. 5, 2025, all three of which are incorporated by reference herein in their entireties.

### FIELD

The disclosure relates to electrical impedance tomography, electrical impedance spectroscopy, electroencephalography, brain-fluid modeling, cerebrospinal-fluid flow modeling, sleep physiology, glymphatic clearance, Bayesian inverse modeling, magnetic-resonance-image registration, digital biomarker development, and computational atlases of intracranial fluid transport.

More particularly, the disclosure concerns systems and methods for constructing a subject-specific or atlas-scaled probabilistic prior model of cerebrospinal-fluid, perivascular, extracellular-space, and parenchymal transport pathways and using that prior model to calibrate high-temporal-density FAST-EIT/FAST-ECS measurements obtained during sleep.

In addition, the functional analysis of brain activity with electroencephalography (EEG) is used to characterize functional components that may index, or are affected by, the measures of cerebral fluid transport.

Finally, the invention may be used for improving the manipulation of brain activity with transcranial electrical stimulation.

### BACKGROUND

Brain clearance physiology involves interactions among cerebrospinal fluid, blood flow, perivascular spaces, extracellular space, parenchymal interstitial fluid, venous outflow, meningeal or lymphatic outflow, and cervical outflow. Clearance-related physiology is modulated by sleep stage, slow oscillations, respiration, cardiac pulsatility, vasomotor oscillations, posture, arousal, autonomic state, and stimulation.

Electrical impedance tomography and spectroscopy can measure changes in electrical conductivity of head and brain tissues. At low frequencies, conductivity is influenced by extracellular ionic conduction and therefore can reflect changes in extracellular-space volume, tortuosity, ionic content, blood volume, and cerebrospinal-fluid-related transport. However, scalp impedance measurements are limited by electrode-skin impedance, scalp conduction, skull resistance, motion, autonomic skin activity, and limited native spatial resolution.

Magnetic resonance imaging can provide anatomical information about brain structure, cerebrospinal-fluid spaces, ventricles, cisterns, white matter, atrophy, vascular injury, and diffusion/perfusion-related biomarkers. MRI may also support registration of electrodes, head tissue conductivity models, DTI-ALPS, IVIM, and other measures relevant to brain-fluid physiology. However, MRI is expensive, episodic, and generally insufficient by itself to provide all-night, sleep-stage-resolved, second-by-second clearance physiology.

A need exists for a method that combines the high anatomical specificity of MRI and brain atlases with the high temporal density of FAST-EIT/FAST-ECS. A further need exists for a probabilistic prior atlas that estimates likely CSF-flow, perivascular, extracellular-space, and parenchymal transport routes for a subject from a T1 MRI or atlas-scaled anatomy, and then calibrates that model using measured EEG/EIT physiology during sleep.

### SUMMARY

In one aspect, the invention provides a computational system that constructs a probabilistic CSF-flow prior atlas in a reference space, registers the atlas to a subject's T1-weighted MRI or atlas-scaled head model, dynamically drives the registered atlas with cardiac, respiratory, vasomotor, sleep-stage, slow-oscillation, and stimulation inputs, and calibrates the resulting predicted extracellular-space and conductivity changes using FAST-EIT or FAST-ECS measurements.

In another aspect, the invention provides a Bayesian super-resolution method in which a high-resolution prior model of plausible intracranial fluid transport is fit to lower-native-resolution boundary impedance measurements. The method estimates a posterior distribution over CSF-flow routes, perivascular transport, extracellular-space expansion, parenchymal exchange, and glymphatic clearance capacity at a spatial scale finer than the native spatial resolution of the impedance measurement.

In another aspect, the invention provides a method of constructing a subject-specific CSF-flow atlas from a T1 MRI by segmenting or registering ventricles, cisterns, subarachnoid spaces, foramen magnum, cervical CSF spaces, vascular territories, perivascular-route priors, parenchymal compartments, venous outflow routes, and extracellular-space susceptibility maps.

In another aspect, the invention provides a four-night or multi-night sleep-assessment protocol in which FAST-ECS measurements are acquired using a wearable WISP device or a high-density EEG/EIT system, physiological driver signals are extracted from EEG/EIT and auxiliary sensors, and dried blood spot or dried plasma spot biomarkers are obtained after the assessment to validate or update a clearance-reserve stage.

In another aspect, the invention provides a physiological staging method that classifies a subject's clearance reserve based on FAST-ECS responses to sleep stages, N2-to-N3 transitions, REM sleep, cardiac pulsatility, respiration, vasomotor oscillations, slow oscillations, arousals, and WISP slow-oscillation transcranial electrical stimulation, and then validates or calibrates that stage using dried blood/plasma spot measures of amyloid, tau, glial activation, neurodegeneration, or synucleinopathy markers.

In another aspect, the invention provides a model-adequacy metric that compares predicted FAST-EIT/FAST-ECS responses from the CSF-flow prior atlas with observed responses during sleep transitions, respiratory cycles, cardiac cycles, vasomotor oscillations, and stimulation/sham challenge windows.

In another aspect, the inclusion of brain functional measures allows the creation of a cortico-hydraulic coupling map for each subject:
1. an MRI-registered cortical source model estimates cortical and subcortically regulated functional states from hdEEG;
2. a probabilistic CSF-flow prior atlas estimates likely CSF, perivascular, parenchymal, and extracellular-space transport pathways;
3. FAST-EIT/FAST-ECS estimates dynamic conductivity and extracellular-space changes during sleep;
4. cardiac, respiratory, vasomotor, sleep-stage, slow-oscillation, REM, arousal, and stimulation signals are modeled as physiological drivers; and
5. Bayesian inference estimates the posterior relationship between localized cortical source patterns and levels, routes, timing, and quality of CSF/ECS flow.

The invention further provides a system and method for selecting, optimizing, and adapting transcranial electrical stimulation to move an individual from a current neurophysiological state toward a selected target state. The system estimates the current state using individualized hdEEG source analysis, FAST-EIT/FAST-ECS, sleep staging, cardiac, respiratory, vasomotor, autonomic, and skin-state measurements. The system compares the current state to a target-state library, predicts the stimulation protocol most likely to produce the desired state transition, delivers stimulation under electrical, sleep, and physiological safety constraints, and uses post-stimulation EEG/EIT response to update the model.

### Definitions

FAST-ECS means Fast-sampled Acquisition and Signal-separation Technology for monitoring Extracellular Space, including any impedance-derived estimate of extracellular-space dynamics obtained from scalp, head, or intracranial electrical measurements.

FAST-EIT means fast-sampled electrical impedance tomography or spectroscopy configured to measure dynamic conductivity changes of the head or brain at high temporal density.

Boundary impedance or bZ means impedance, transfer impedance, admittance, conductivity, or voltage response data measured between or among scalp electrodes after current injection.

CSF-flow prior atlas means a probabilistic computational atlas representing likely CSF, perivascular, extracellular-space, parenchymal, venous, meningeal, cervical, or lymphatic transport routes, together with driver-specific gain, lag, compliance, conductance, or uncertainty parameters.

Driver means a physiological or therapeutic signal that influences fluid transport, including cardiac pulsation, respiration, vasomotor oscillation, sleep stage, slow oscillation, REM phasic activity, arousal, posture, autonomic state, or transcranial stimulation.

Bayesian super-resolution means estimating a posterior distribution over high-resolution anatomical or physiological state variables using lower-resolution observations constrained by a high-resolution prior model.

DBPS means dried blood spot, dried plasma spot, dried capillary blood, dried venous blood, or equivalent dried microsample matrix used to measure biomarkers.

Clearance reserve stage means a physiologic classification of a subject's sleep-dependent brain-clearance capacity based on FAST-ECS and related driver-response features, optionally validated or updated by DBPS or plasma biomarkers.

Glymphatic Clearance Capacity or GCC means a scalar, vector, map, stage, trajectory, posterior distribution, or report representing the subject's inferred ability to exchange CSF and interstitial fluid and clear solutes during sleep or other physiological states.

Cortico-hydraulic coupling means a quantified relationship between one or more cortical source-localized EEG patterns and one or more CSF, perivascular, extracellular-space, parenchymal, or glymphatic-clearance state variables.

Neurohydraulic state means a combined neural and fluid-transport state comprising cortical source activity, sleep state, autonomic state, blood-flow state, CSF-flow state, extracellular-space state, and clearance state.

Source-resolved sleep driver means a sleep-related EEG feature localized to the subject's cortical surface, including slow oscillation source amplitude, slow oscillation phase, propagation direction, spindle coupling, REM phasic activity, beta/arousal activity, or infraslow activity.

Cortico-hydraulic coupling matrix means a matrix, tensor, graph, or probabilistic map linking cortical source regions and time lags to CSF-flow, ECS, FAST-EIT, or clearance-related regions.

Flow quality means a classification of CSF/ECS transport dynamics including coherent, respiratory-locked, cardiac-locked, vasomotor-locked, slow-oscillation-locked, delayed, fragmented, uncoupled, arousal-suppressed, stimulation-responsive, or stimulation-nonresponsive flow.

Target state means a desired neurophysiological, neurohydraulic, sleep, autonomic, cognitive, affective, behavioral, or therapeutic state represented by one or more measurable features of EEG, source-localized EEG, EIT, FAST-ECS, respiration, cardiac activity, vasomotor activity, sleep stage, biomarkers, or self-report.

Target-state library means a database or model containing one or more desired states, each represented by physiological feature ranges, source-space patterns, impedance/ECS patterns, stimulation-response signatures, safety constraints, and target-engagement criteria.

State-transition model means a model that predicts how stimulation parameters alter the current state of the subject toward a target state.

Stimulation policy means a rule, algorithm, Bayesian decision function, model-predictive controller, reinforcement-learning policy, or optimization procedure that selects stimulation montage, waveform, amplitude, frequency, phase, timing, duty cycle, duration, and stopping criteria.

Neurohydraulic state means a combined estimate of neural source activity, brain conductivity, extracellular-space state, CSF/ECS transport, sleep stage, respiration, cardiac activity, vasomotor state, autonomic state, and stimulation responsiveness.

Affective target state means a measurable physiological and optionally self-reported state associated with calm, positive affect, reduced anxiety, reduced rumination, reduced hyperarousal, improved emotional regulation, or related user- or clinician-selected outcomes.

### BRIEF DESCRIPTION OF THE DRAWINGS

In these figures, solid arrows show measured data or generated outputs. Dashed arrows show calibration or feedback.

FIG. 1: The invention is a unified WISP/hdEEG/EIT/tES platform for measuring, modeling, staging, and modulating sleep-dependent brain clearance. The figure illustrates alternative wearable and high-density electrode embodiments that acquire EEG, boundary impedance, stimulation, and physiological driver signals during sleep. These signals are registered to an anatomical/modeling workspace that includes a subject-specific or atlas-scaled head model, a CSF-flow prior atlas, cortical source analysis, an electrical forward model, and a Bayesian superresolution engine. The outputs include FAST-ECS, GCC/GCI/RGCI, clearance-reserve stage, DBPS validation, target engagement, and model update metrics.

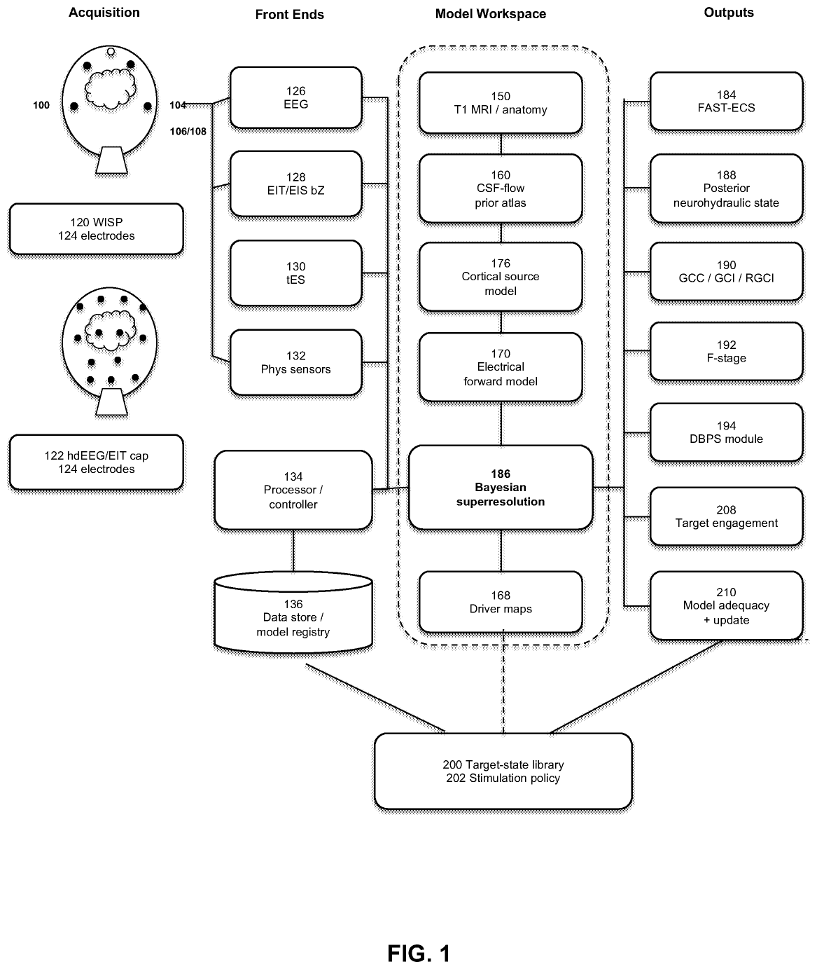

FIG. 2: MRI and atlas data create a subject-specific probabilistic CSF/PVS/ECS prior, including vasculature, outflow, driver maps, and uncertainty. The figure illustrates how T1 MRI, high-resolution T2/PVS imaging, TOF-MRA, SWI/QSM, diffusion/IVIM, and a reference CSF-flow atlas may be nonlinearly registered into the subject's anatomical space. The resulting model estimates likely ventriculo-cisternal, subarachnoid, perivascular, parenchymal, venous, meningeal, foramen-magnum, and cervical outflow routes. Driver-specific maps for cardiac pulsatility, respiration, vasomotor oscillation, sleep stage, slow oscillations, REM activity, arousal, and stimulation are represented as probabilistic priors with associated uncertainty.

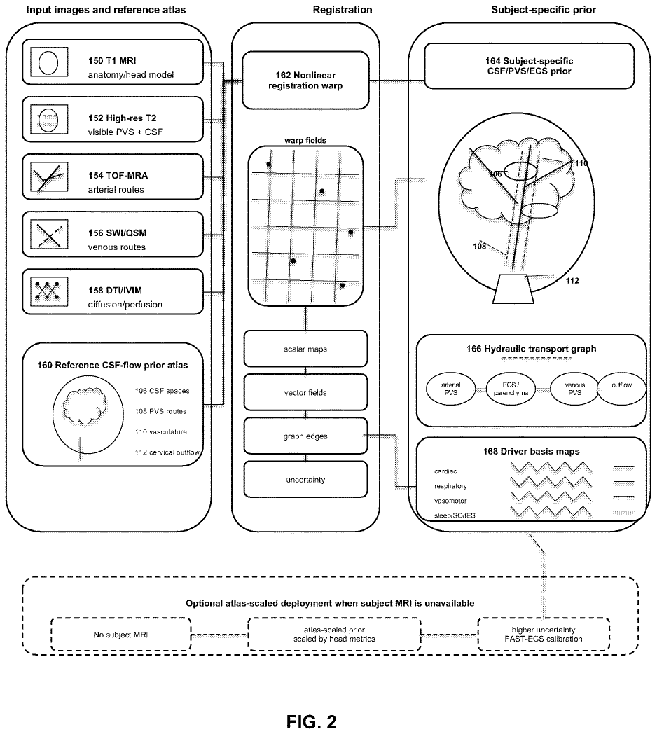

FIG. 3: FAST-EIT/FAST-ECS becomes superresolved by fitting low-resolution boundary impedance to a high-resolution anatomical-hydraulic prior. The figure illustrates a Bayesian loop in which a high-resolution CSF/PVS/ECS prior predicts conductivity and extracellular-space changes, an electrical forward model predicts boundary impedance, and observed skin-path-corrected impedance updates the posterior state estimate. The posterior includes probable CSF-flow routes, perivascular exchange, ECS expansion, parenchymal transport, GCC/GCI/RGCI, and uncertainty. The figure also shows model-adequacy testing by comparing predicted and observed held-out FAST-EIT/FAST-ECS dynamics.

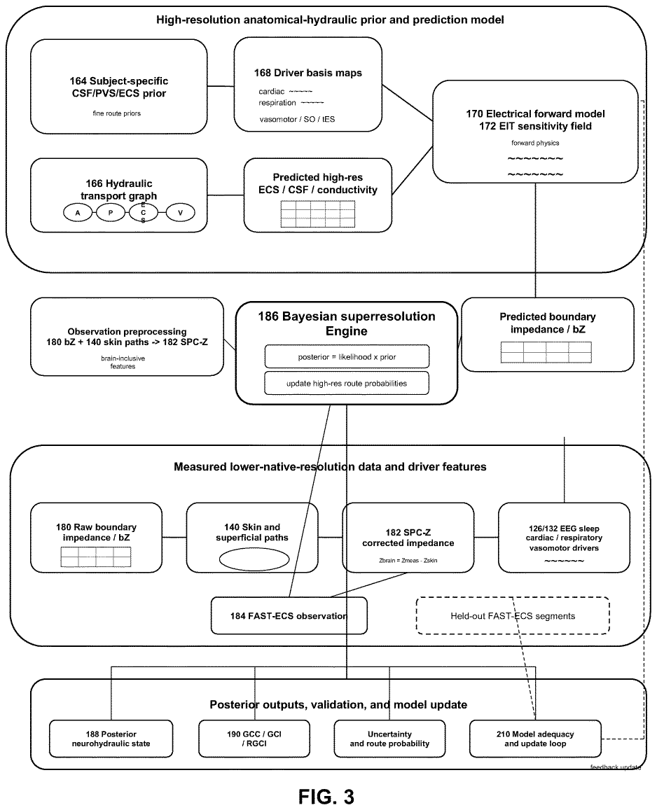

FIG. 4: Source-localized cortical sleep physiology is coupled to regional CSF/ECS dynamics to classify the quality of brain clearance. The figure illustrates MRI-registered hdEEG source analysis in which slow oscillations, REM activity, arousal/beta activity, spindle coupling, theta activity, and infraslow cortical activity are localized to individual cortical source regions. These source-resolved neural drivers are compared with FAST-ECS and CSF/PVS posterior estimates across physiological time lags. The system outputs cortico-hydraulic coupling maps or matrices and classifies flow quality as coherent, delayed, fragmented, uncoupled, arousal-suppressed, stimulation-responsive, or stimulation-nonresponsive.

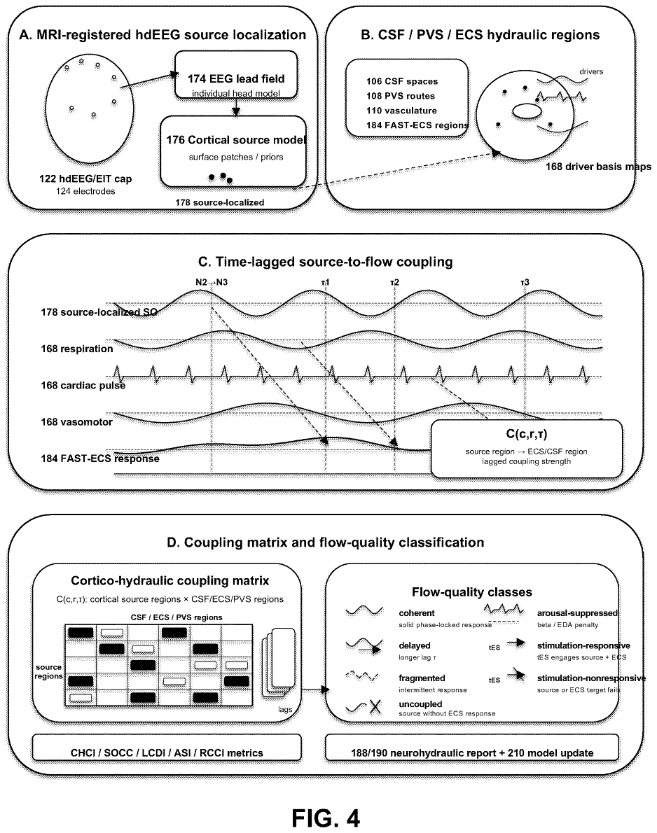

FIG. 5: tES is selected from a subject-specific neurohydraulic digital twin, delivered under safety constraints, and validated by EEG/EIT target engagement. The figure illustrates a closed-loop control architecture in which the current neurohydraulic state is estimated from cortical source activity, FAST-ECS, sleep stage, cardiac, respiratory, vasomotor, skin-state, and GCC features. A target-state library and stimulation policy select a candidate tES protocol predicted to move the subject toward a desired sleep, clearance, autonomic, cognitive, affective, or therapeutic state while preserving electrical and sleep-architecture safety. Post-stimulation EEG and impedance responses are then compared with the predicted response to compute neural target engagement, hydraulic target engagement, sham-adjusted response, model adequacy, and stimulation-policy updates.

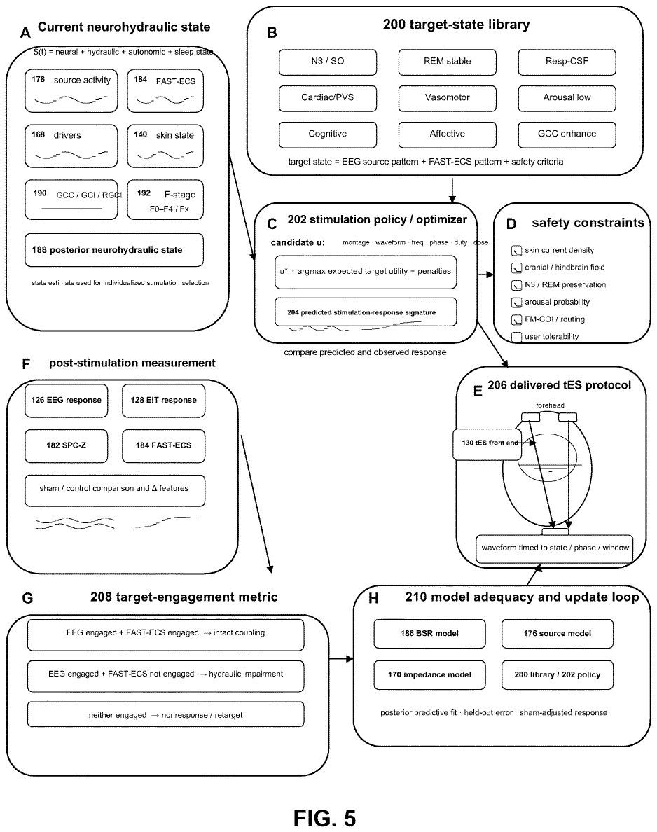

### FIGURE REFERENCE NUMERALS

### Numeral Element

100 Human subject
102 Head/scalp
104 Brain/cortex
106 CSF spaces: ventricles, cisterns, subarachnoid space
108 Perivascular-space/PVS routes
110 Arterial and venous vasculature
112 Foramen magnum/cervical CSF outflow
120 Wearable WISP system
122 High-density EEG/EIT cap or headweb
124 Electrode array
126 EEG acquisition front end
128 EIT/EIS/boundary impedance acquisition front end
130 tES stimulation front end
132 Physiological sensors: respiration, cardiac, motion, EDA
134 Processor/controller
136 Data store/model registry/cloud
140 Skin/superficial impedance paths
150 T1 MRI/subject anatomical model
152 High-resolution T2/PVS image
154 TOF-MRA arterial image
156 SWI/QSM venous image
158 Diffusion/IVIM/DTI image
160 Reference CSF-flow prior atlas
162 Nonlinear registration/atlas-to-subject warp
164 Subject-specific CSF/PVS/ECS prior
166 Hydraulic transport graph
168 Driver basis maps: cardiac, respiratory, vasomotor, sleep, tES
170 Electrical forward model
172 EIT sensitivity field
174 EEG lead field
176 Cortical source model
178 Source-localized hdEEG activity
180 Raw boundary impedance/bZ
182 Skin-path-corrected impedance/SPC-Z
184 FAST-ECS/extracellular-space estimate
186 Bayesian superresolution engine
188 Posterior neurohydraulic state
190 GCC/GCI/RGCI output
192 Clearance-reserve stage/F-stage
194 DBPS biomarker module
200 Target-state library
202 Stimulation policy/optimizer
204 Predicted stimulation-response signature
206 Delivered tES protocol
208 Target-engagement metric
210 Model adequacy and update loop

### DETAILED DESCRIPTION

### 1. System Overview

A system includes one or more of: a scalp electrode array, an EEG acquisition subsystem, an electrical impedance acquisition subsystem, a stimulation subsystem, physiological sensors, a processor, a memory, a T1 MRI or atlas-scaled anatomical model, a probabilistic CSF-flow prior atlas, and a Bayesian calibration engine.

The electrode array may be a high-density cap comprising about 128, 256, 280, or more electrodes, or may be a reduced-channel wearable device such as a WISP headband. In some embodiments, the same electrodes are used for EEG, impedance acquisition, stimulation, electrode-quality monitoring, and skin-state estimation. In other embodiments, separate electrodes are used for stimulation and impedance acquisition.

The processor receives EEG, impedance, stimulation, motion, posture, respiration, cardiac, and skin-state data. The processor extracts driver signals, predicts fluid-transport and extracellular-space responses using the subject-specific CSF-flow prior atlas, computes predicted boundary impedance from the electrical forward model, and updates the model by Bayesian inference using observed FAST-EIT or FAST-ECS data.

### 2. Anatomical Prior Atlas

The prior atlas is constructed in a reference anatomical coordinate system and includes scalar maps, vector maps, compartment labels, graph edges, tissue priors, and uncertainty maps.

The atlas may include:
lateral ventricles;
third ventricle;
fourth ventricle;
cerebral aqueduct;
basal cisterns;
cisterna magna;
convexity subarachnoid space;
cerebellar and brainstem CSF spaces;
foramen magnum;
upper cervical CSF spaces;
choroid plexus regions;
arteries and arterial territories;
veins and venous sinuses;
perivascular-route probability maps;
periventricular white matter;
deep white matter;
corpus callosum;
superior longitudinal fasciculus;
hippocampal and perihippocampal regions;
medial temporal regions;
orbitofrontal and limbic regions;
posterior cingulate and precuneus;
meningeal, dural, cervical, or lymphatic outflow regions.

The prior atlas may be built from public or private imaging datasets, vessel atlases, CSF-space segmentations, histological priors, diffusion MRI priors, vascular territory maps, sleep MRI studies, computational fluid dynamics simulations, perivascular-space studies, or expert-curated anatomical rules.

### 3. Transport Graph

The prior atlas may be represented as a graph in which nodes correspond to fluid or tissue compartments and edges correspond to probable transport paths.

Example node classes include:
arterial input nodes;
periarterial perivascular nodes;
CSF reservoir nodes;
parenchymal ECS nodes;
perivenous route nodes;
venous sinus nodes;
meningeal or lymphatic nodes;
cervical outflow nodes;
biomarker appearance nodes.

An edge between nodes may have a conductance, compliance, delay, flow direction, driver sensitivity, solute-transfer rate, or uncertainty parameter. For example:

Q_ij(t)=G_ij(P_i(t)−P_j(t))+D_ij(t)+ε_ij(t)

where Q_ij is flow from node i to node j, G_ij is hydraulic conductance, P_i−P_j is a pressure difference, D_ij (t) is a driver-dependent term, and ε_ij is uncertainty.

### 4. Driver-Specific Basis Maps

The prior atlas includes driver-specific dynamic basis maps. These maps predict which regions or pathways are likely to respond to a physiological driver, with what amplitude and lag.

### Cardiac Driver

A cardiac driver map represents pulse-driven arterial, perivascular, CSF, and parenchymal effects. It may be derived from ECG, PPG, EEG cardiac artifact, EIT cardiac components, or pulse oximetry.

The cardiac response may be represented as:

Φ_cardiac(r,t)=A_c(r)h_c(t−τ_c(r))
where A_c(r) is a regional cardiac gain, h_c is a pulse waveform, and τ_c(r) is a regional lag.

### Respiratory Driver

A respiratory driver map represents effects of thoracic pressure, venous pressure, diaphragmatic motion, CSF displacement, and respiratory-linked CSF inflow. It may be derived from respiratory belts, nasal airflow, impedance respiration, EEG respiratory artifact, or EIT respiratory components.

The respiratory response may be represented as:

Φ_resp(r,t)=A_r(r)h_r(t−τ_r(r))

### Vasomotor Driver

A vasomotor driver map represents low-frequency vascular oscillations, blood-volume oscillations, and perivascular fluid transport. It may include infraslow frequency bands such as about 0.01-0.20 Hz or about 0.05-0.15 Hz.

The vasomotor response may be represented as:

Φ_vaso(r,t)=A_v(r)h_v(t−τ_v(r)

### Sleep-Stage and Slow-Oscillation Driver

A sleep-stage driver map represents N2, N3, REM, wake, arousal, N2-to-N3 transition, REM phasic activity, slow oscillation phase, slow oscillation amplitude, spindle activity, and NREM-REM cycling

The sleep response may be represented as:

Φ_sleep(r,t)=A_s(r)S_stage(t)+A_SO(r)SO(t−τ_SO(r))+A_REM(r)REM_phasic(t−τ_REM(r))

### Stimulation Driver

A stimulation driver map represents transcranial electrical stimulation, acoustic stimulation, respiratory pacing, posture change, or other controlled perturbation. For tES, the driver map may include electric field, current density, electrode montage, stimulation phase, stimulation frequency, duty cycle, stimulation amplitude, and predicted current occupancy through a target anatomical region such as the foramen magnum.

### 5. Subject-Specific Registration to T1 MRI

For a subject having a T1 MRI, the processor segments the head and brain and registers the prior atlas to subject space.

The segmentation may include:
scalp;
skull;
CSF;
gray matter;
white matter;
ventricles;
cerebellum;
brainstem;
eyes;
air spaces;
cervical structures;
major vascular or sinus structures if visible.

A nonlinear diffeomorphic registration maps the reference atlas into the subject's native MRI space. Scalar probability maps are warped to subject space. Vector flow fields are transformed using the Jacobian of the deformation. Conductance and compliance parameters may be scaled by local deformation, CSF-space volume, ventricular size, age, atrophy, white-matter hyperintensity burden, or head size.

For subjects without MRI, the atlas may be scaled using head circumference, cranial landmarks, electrode positions, age, sex, anthropometrics, impedance-transfer measurements, or cohort priors. In such embodiments, the system outputs higher uncertainty.

### 6. Electrode Registration and Electrical Forward Model

For high-density EEG/EIT, electrode positions are registered to the subject's scalp surface using photogrammetry, digitization, cap geometry, fiducials, or image-based registration. For WISP or reduced-channel devices, electrode locations are estimated from device geometry, landmarks, or calibration measurements.

The electrical forward model solves for voltage or admittance responses to current injection through the electrode array. The model may include scalp, skull, CSF, gray matter, white matter, skin-electrode interface, gel, sweat, electrodermal activity, and electrode motion.

The EIT observation equation may be:

ΔZ_k(t)=Σ_r S_k(r)Δσ(r,t)+S_skin,k(t)+ε_k(t)

where ΔZ_k (t) is the measured change for impedance pattern k, S_k(r) is regional EIT sensitivity, Δσ(r,t) is regional conductivity change, S_skin,k(t) is superficial/skin contribution, and ε_k(t) is noise.

Conductivity may be related to extracellular-space state by:

Δσ(r,t)=β1(r)ΔECS(r,t)+β2(r)Δblood(r,t)+β3(r)ΔCSF(r,t)+β4(r)Δion(r,t)+β5(r)artifact(r,t)

### 7. Skin and Superficial-Path Correction

The system estimates and removes or models skin, scalp, electrode, sweat, and superficial-path artifacts.

For WISP-like embodiments, the processor may compute skin-path-corrected intracranial impedance:

SPC-Z=Z_meas−αZ_skin1−βZ_skin2−γZ_skin3
where Z_meas is a brain-inclusive impedance path and Z_skin1, Z_skin2, and Z_skin3 are skin-only or superficial-control paths. Weights may be fixed, stage-conditioned, subject-specific, learned from same-night data, learned across historical nights, or jointly estimated in the Bayesian model.

For high-density embodiments, superficial artifacts may be estimated using spatial covariance, local adjacent-pair measurements, electrodermal activity, current-density sensitivity, temporal signatures, independent components, regression, adaptive filtering, model-based inversion, or Bayesian source separation.

### 8. Bayesian Super-Resolution Calibration

The system estimates a posterior distribution over high-resolution CSF-flow, ECS, and clearance states using lower-resolution EIT/FAST-ECS data.

Let:
x_H(t)=high-resolution hydraulic state, including CSF flow, perivascular flow, ECS fraction, parenchymal exchange, blood volume, solute movement, and clearance flux.
y_Z(t)=measured FAST-EIT or FAST-ECS boundary impedance.
y_EEG(t)=measured EEG and sleep features.
d(t)=physiological drivers.
A_T1=subject-specific registered atlas.

The posterior may be:

p(x_H(t),θ|y_Z,y_EEG,d,A_T1)∝p(y_Z|x_H,θ,A_T1)p(x_H|d,A_T1,0)p(θ)

The posterior may be computed using variational Bayes, Markov chain Monte Carlo, particle filtering, ensemble Kalman filtering, expectation-maximization, differentiable simulation, neural posterior estimation, or combinations thereof.

The super-resolution result is not a claim that the raw impedance instrument directly resolves every microscopic pathway. Rather, the high-resolution atlas constrains the plausible physiological state space, and the lower-resolution FAST-EIT/FAST-ECS data update the probability of high-resolution routes and regional states.

### 9. Four-Night FAST-ECS Protocol

In some embodiments, a subject completes a multi-night assessment, such as four nights.

For WISP participants:
Night 1 may be baseline or adaptation.
Night 2 may be active or sham stimulation.
Night 3 may be the opposite stimulation condition.
Night 4 may be active, sham, baseline, individualized target engagement, or model-calibration night.

For hdEEG/EIT participants:
One or more nights may be high-density all-night recording.
The system may acquire 128, 256, 280, or more EEG/EIT channels.
High-density data may calibrate a reduced-channel wearable model.

Across nights, the system extracts:
sleep stage;
N2-to-N3 transition timing;
N3 slow oscillation phase and amplitude;
REM phasic activity;
cardiac phase and amplitude;
respiratory phase and amplitude;
vasomotor oscillation phase and amplitude;
arousals;
motion;
posture;
skin-state signals;
stimulation or sham timing;
FAST-ECS state variables.

### 10. DBPS Biomarker Calibration

After a multi-night assessment, such as after each four-night block, a dried blood spot, dried plasma spot, venous plasma sample, capillary plasma sample, or other microsample may be collected.

Analytes may include:
Aβ40;
Aβ42;
Aβ42/Aβ40 ratio;
p-tau217;
p-tau181;
total tau;
GFAP;
NfL;
α-synuclein;
phosphorylated α-synuclein;
oligomeric or aggregate α-synuclein;
inflammatory markers;
vascular markers;
metabolic markers.

The biomarker measurements may be used to validate, calibrate, or update slower parameters of the clearance model. The biomarkers need not be treated as instantaneous labels for a single night. Instead, they may update estimates of chronic molecular burden, acute efflux, clearance reserve, disease progression state, peripheral clearance, blood-brain barrier transfer, or model uncertainty.

### 11. Clearance-Reserve Staging

The system may classify a subject into a clearance-reserve stage using FAST-ECS and physiological driver responses.

One example stage model is:

### F0: Normal Clearance Reserve

Robust overnight FAST-ECS change, robust N2-to-N3 and REM responses, preserved cardiac/respiratory/vasomotor coupling, and normal biomarker pattern.

F1: Sleep-Driver Insufficiency with Preserved Reserve

Reduced sleep opportunity or fragmented N3/REM, but preserved FAST-ECS response when sleep drivers occur; stimulation produces robust target engagement.

### F2: Early Clearance-Reserve Failure/Amyloid-Risk Stage

Blunted N2-to-N3 FAST-ECS response, reduced driver coupling, or delayed ECS expansion despite adequate sleep opportunity; DBPS may show abnormal or trending amyloid ratio.

### F3: Tau-Activation Stage

Persistent impaired FAST-ECS response with p-tau positivity or rising tau-related measures; stimulation may increase EEG slow-wave activity without adequate FAST-ECS response.

### F4: Neurodegenerative Clearance Failure

Minimal FAST-ECS response to sleep drivers or stimulation, poor coupling, high arousal burden, and biomarker evidence of neurodegeneration such as elevated NfL.

### Fx: Mixed or Non-AD Clearance Impairment

FAST-ECS impairment with non-AD or mixed biomarker pattern, such as vascular, inflammatory, respiratory, medication-related, autonomic, or synucleinopathy-linked impairment.

The stage labels are examples. Other labels, scores, or probabilistic categories may be used.

### 12. Stimulation as Target Engagement and Model Adequacy

In some embodiments, the system delivers or schedules stimulation during sleep. The stimulation may include slow-oscillation tES at about 0.1-2 Hz, including about 0.75 Hz, delivered during an N2-to-N3 transition window or predicted glymphatic-clearance window.

Before stimulation, the system predicts an expected response:

p(ΔFAST-ECS,ASWA,ΔN3,ΔRGCI|u,x_H,θ)
where u is stimulation amplitude, frequency, phase, montage, duty cycle, timing, or dose.

After stimulation, the system measures the observed response and computes:
EEG target engagement;
impedance target engagement;
regional GCC response;
sham-adjusted response;
posterior predictive accuracy;
BSR adequacy;
stimulation-policy update.

The system may detect cases in which EEG target engagement occurs without FAST-ECS target engagement, indicating impaired downstream clearance reserve.

### 13. Model Adequacy and Quality Control

The system may compute model adequacy by comparing predicted and observed FAST-ECS dynamics.

Adequacy may be measured using:
posterior predictive likelihood;
held-out prediction error;
temporal lag error;
spatial correlation between predicted and observed hdEIT maps;
respiratory phase-locking accuracy;
cardiac phase-locking accuracy;
vasomotor coherence;
N2-to-N3 response accuracy;
REM response accuracy;
active-versus-sham response accuracy;
DBPS agreement;
high-density-to-WISP transfer accuracy.

A BSR adequacy score may be:

A_BSR=log p(y_heldout|CSF-prior BSR model)−log p(y_heldout|low-resolution EIT-only model)

If adequacy is low, the system may increase uncertainty, request a high-density calibration, revise skin-path correction, modify driver gains, update conductance parameters, exclude low-quality epochs, or report that the model is not adequate for subject-specific staging.

### 14. Outputs

The system may output:
subject-specific CSF-flow prior map;
posterior CSF-flow route probability map;
posterior ECS expansion map;
regional FAST-ECS map;
regional GCC map;
global GCC score;
clearance reserve stage;
driver-response profile;
respiratory coupling index;
cardiac coupling index;
vasomotor coupling index;
slow-oscillation coupling index;
N2-to-N3 clearance response;
REM clearance response;
tES target engagement score;
BSR adequacy score;
DBPS-calibrated molecular stage probability;
longitudinal risk score;
recommendation for high-density calibration or wearable monitoring.

### 15. Example Embodiments

### Example 1—T1-Registered CSF-Flow Prior Atlas

A subject undergoes T1 MRI. The system segments the subject's head and registers a reference CSF-flow prior atlas to the subject's anatomy. The registered atlas estimates likely CSF reservoirs, perivascular routes, parenchymal exchange regions, and cervical outflow pathways.

The system generates driver-specific maps for cardiac, respiratory, vasomotor, N2-to-N3, REM, and slow-oscillation physiology.

### Example 2—Four-Night WISP FAST-ECS Assessment

A subject completes four nights with a WISP device. EEG and impedance are recorded during sleep. Sleep stage, slow oscillations, respiration, cardiac timing, vasomotor oscillations, and skin-path-corrected impedance are extracted. A morning dried plasma spot is obtained after the fourth night. The system estimates clearance reserve stage and compares the stage with Aβ42/Aβ40, p-tau217, GFAP, and NfL.

### Example 3—High-Density EEG/EIT Calibration

A subject completes a high-density 280-channel EEG/EIT night. Electrode positions are registered to the subject's T1 MRI. The electrical forward model computes regional EIT sensitivity maps. The CSF-flow prior atlas predicts driver-locked conductivity changes. The Bayesian calibrator updates subject-specific driver gains, lags, and regional ECS parameters.

### Example 4—Stimulation Challenge

A subject receives slow-oscillation tES during an N2-to-N3 transition window. The model predicts increased slow-wave activity and decreased skin-path-corrected impedance. If the EEG response occurs but the impedance response is absent, the system classifies the subject as having impaired clearance reserve despite preserved sleep-neural target engagement.

### Example 5—Atlas-Only Deployment

A subject lacks MRI. The system scales the atlas by head size, electrode geometry, age, sex, and impedance-transfer measurements. The system outputs a lower-confidence CSF-flow prior and calibrates it using four nights of FAST-ECS data.

Cortically Localized hdEEG Source Analysis for Neurohydraulic Modeling

In some embodiments, the system uses high-density EEG source analysis to characterize the subject's cortical functional state and to associate that state with levels and qualities of CSF, perivascular, extracellular-space, and parenchymal flow.

A subject-specific head model is constructed from T1-weighted MRI, optionally with diffusion MRI tractography. Electrode positions are registered to the individual scalp surface using photogrammetry or digitization. The head model includes scalp, skull, CSF, gray matter, white matter, internal air, eyes, and other tissue compartments. The same model may be used for EEG lead-field computation, EIT sensitivity-field computation, and stimulation current-flow modeling.

The cortical surface is segmented and tessellated into source patches. In some embodiments, the native source model includes thousands of equivalent dipole sources. In other embodiments, a Bayesian super-resolution model subdivides native cortical patches into macrocolumn-level or other high-resolution source elements using anatomical, tractographic, and physiological priors. This is consistent with the prior Bayesian super-resolution architecture in which a lower-resolution hdEEG source posterior is used to inform a higher-resolution prior for individualized cortical modeling.

The source-localized EEG model estimates one or more of:
slow oscillation source amplitude;
slow oscillation phase;
slow oscillation polarity inversion;
slow oscillation propagation direction;
slow oscillation source region;
spindle amplitude and phase;
spindle-slow oscillation coupling;
REM phasic source activity;
theta activity;
beta/arousal activity;
infraslow cortical activity;
source-space coherence;
source-space phase synchrony;
source-space traveling waves;
task-evoked or memory-related source activity;
subcortical regulatory latent variables inferred from cortical activity variance.

These source features are then entered into the hydraulic model as neural drivers or state variables.

### Neural Source Drivers of CSF/ECS Transport

The system models neural activity as one class of driver for fluid transport. Neural source activity may influence or organize CSF/ECS transport through sleep-stage transitions, vascular tone, respiration coupling, autonomic state, slow oscillation timing, local metabolic demand, and arousal suppression.

For a cortical source region c and a fluid or ECS region r, the system may estimate:

ECS_r(t)=β0(r)+ΣcβSO(c,r,τ)SO_c(t−τ)+βREM(c,r,τ)REM_c(t−τ)+ββ(c,r,τ)Beta_c(t−τ)+βresp(r,t)Resp(t−τ)+βcard(r,τ)Card(t−τ)+βvaso(r,τ)Vaso(t−τ)+ε

where:
SO_c(t) is source-localized slow oscillation activity in cortical source region c;
REM_c(t) is source-localized REM phasic activity;
Beta_c(t) is source-localized beta or arousal activity;
Resp (t) is respiratory phase or amplitude;
Card (t) is cardiac phase or pulse amplitude;
Vaso (t) is vasomotor oscillation;
τ is a physiologically plausible time lag;
ECS_r(t) is the inferred extracellular-space or FAST-ECS state in region r.

The coefficients define a cortico-hydraulic coupling matrix:

C(c,r,τ)=influence or association of cortical source c on hydraulic/ECS region r at lagτ

This matrix may be estimated for each subject and then compared across subjects, disease stages, sleep stages, stimulation conditions, and biomarker profiles.

### Source-Resolved Sleep Clearance Phenotypes

The system may classify the quality of CSF/ECS flow by combining localized cortical source features with FAST-EIT/FAST-ECS responses.

### High-Quality Clearance Phenotype

A high-quality clearance phenotype may include:
stable N2-to-N3 transition;
source-localized slow oscillations with strong limbic, medial temporal, caudal orbital, or other sleep-regulatory generators;
coherent slow oscillation propagation;
strong coupling of slow oscillation phase to respiratory and vasomotor rhythms;
robust decrease in skin-path-corrected impedance;
increased FAST-ECS in predicted CSF/ECS regions;
preserved REM with controlled electrodermal artifact;
strong stimulation target engagement when WISP SO-tES is applied.

### Sleep-Driver Insufficiency Phenotype

A sleep-driver insufficiency phenotype may include:
reduced N3 or fragmented N3;
insufficient slow oscillation amplitude or continuity;
preserved FAST-ECS response when adequate slow oscillations occur;
good response to WISP SO-tES;
relatively preserved DBPS biomarker profile.

### Neural-Hydraulic Uncoupling Phenotype

A neural-hydraulic uncoupling phenotype may include:
apparently adequate N3 or slow oscillation activity by EEG;
weak or delayed FAST-ECS response;
poor source-to-ECS coupling;
weak respiratory or cardiac coupling;
possible early amyloid-clearance risk.

### Arousal-Suppressed Clearance Phenotype

An arousal-suppressed phenotype may include:
increased beta or high-frequency arousal sources;
elevated sympathetic/EDA signatures;
reduced N3 continuity;
increased heart-rate burden;
diminished FAST-ECS despite sleep-stage labels suggesting NREM sleep.

### Stimulation-Nonresponsive Phenotype

A stimulation-nonresponsive phenotype may include:
WISP SO-tES increases slow-wave activity but does not produce the expected FAST-ECS decrease in impedance;
or WISP SO-tES fails to increase either slow-wave source engagement or FAST-ECS;
this pattern may indicate impaired clearance reserve or inaccurate stimulation targeting.

This staging can be mapped to DBPS molecular findings, including Aβ42/Aβ40, p-tau217, GFAP, and NfL.

### Source-Resolved Flow Quality Metrics

The system may output the following metrics:
Cortico-Hydraulic Coupling Index, CHCI

A scalar or regional index measuring how strongly localized cortical source activity predicts FAST-ECS or CSF-flow state.

### Slow-Oscillation Clearance Coupling, SOCC

The degree to which source-localized slow oscillations produce or predict expected ECS expansion.

### Limbic Clearance Driver Index, LCDI

The degree to which limbic, medial temporal, caudal orbital, or other sleep-regulatory sources are associated with clearance-favorable FAST-ECS dynamics.

### Arousal Suppression Index, ASI

The degree to which source-localized beta/arousal patterns suppress or disrupt CSF/ECS flow.

### REM Clearance Coupling Index, RCCI

The degree to which REM source patterns predict FAST-ECS changes after correction for skin and electrodermal artifacts.

### Stimulation Coupling Gain, SCG

The degree to which tES-induced changes in cortical source activity produce the predicted FAST-ECS response.

### Neurohydraulic Adequacy Score, NHAS

A posterior predictive score measuring whether the subject's cortical source dynamics and FAST-ECS dynamics are mutually consistent under the CSF-flow prior atlas.

Integration with the CSF-Flow Prior Atlas

The CSF-flow prior atlas estimates anatomically likely CSF, perivascular, parenchymal, and extracellular-space routes. The hdEEG source model adds a functional layer to that atlas.

The combined prior may be expressed as:

p(x_H,x_N,θ|T1,atlas)

where:
x_H is the hydraulic state;
x_N is the neural source state;
θ includes coupling parameters;
T1 is the subject-specific MRI;
atlas is the probabilistic CSF-flow prior atlas.

The posterior becomes:

p(x_H,x_N,θ|FAST-EIT,hdEEG,drivers,T1,atlas)

This allows the system to estimate not only where flow is likely, but also which functional cortical patterns make the flow likely, absent, delayed, fragmented, or stimulation-responsive.

Integration with WISP and hdEEG/EIT

In high-density laboratory embodiments, 128-, 256-, 280-, or higher-channel hdEEG/EIT is used to estimate detailed cortical sources and regional FAST-ECS maps. The GCC grant materials already describe use of 256/280-channel EEG, photogrammetry, individual MRI registration, Sourcerer head modeling, and source localization of slow oscillations.

In reduced-channel WISP embodiments, the high-density source-flow model may be compressed into a lower-dimensional wearable model. The wearable model may estimate proxy source states such as:
N2-to-N3 transition probability;
slow oscillation amplitude and phase;
REM phasic probability;
arousal/beta burden;
respiratory and cardiac drivers;
stimulation response;
WISP FAST-ECS response.

A prior high-density session may train the WISP model for the individual subject. Alternatively, population priors may be used when no MRI or high-density calibration is available.

### Stimulation as a Source-to-Flow Challenge Test

The system may use WISP SO-tES or hd-tES as an active perturbation to test neurohydraulic coupling.

The stimulation model predicts:

p(ΔSource_c,ΔFAST-ECS_r|stimulation u,current-flow model,source prior,hydraulic prior)

After stimulation, the system measures:
whether the intended cortical source pattern changed;
whether the expected FAST-ECS response occurred;
whether the response occurred in the expected region and lag;
whether the subject showed EEG target engagement without hydraulic target engagement.

This creates a causal test of whether the BSR model is adequate. The existing WISP results already show that slow-oscillation tES increased N3/REM sleep and produced stimulation-related impedance reduction around N2-to-N3 transition, making this a strong target-engagement embodiment.

### Adaptive Target-State Neuromodulation

In some embodiments, the system is not limited to slow-oscillation stimulation during N2-to-N3 sleep. Instead, the system selects from a library of target states and computes a subject-specific stimulation policy to move the subject toward the selected state.

The target state may be selected by a clinician, user, protocol, algorithm, biomarker stage, sleep stage, current neurohydraulic state, or safety controller. The target state may include, without limitation:
1. N3 slow-oscillation enhancement;
2. N2-to-N3 transition facilitation;
3. REM stabilization;
4. NREM-REM cycling optimization;
5. arousal suppression;
6. sympathetic/autonomic calming;
7. respiratory-CSF coupling enhancement;
8. cardiac/perivascular coupling enhancement;
9. vasomotor/infraslow organization;
10. memory-consolidation enhancement;
11. attention/readiness enhancement;
12. affective regulation;
13. anxiety or hyperarousal reduction;
14. cognitive recovery after sleep deprivation;
15. clearance-favorable GCC enhancement;
16. stimulation nonresponse detection;
17. avoidance of an adverse state such as sleep fragmentation, excessive arousal, REM suppression, or autonomic activation.

The target-state library may represent each state as a vector:

T_j={EEG_source_pattern,FAST-ECS_pattern,sleep_features,autonomic_features,respiratory_features,cardiac_features,vasomotor_features,subjective_features,safety_constraints,target_engagement_criteria}

The current state is estimated as:

S(t)={x_N(t),x_H(t),x_A(t),x_sleep(t),x_skin(t),x_safety(t)}

where x_N is cortical source state, x_H is hydraulic/ECS state, x_A is autonomic/respiratory/cardiac state, x_sleep is sleep-stage state, x_skin is skin/electrode artifact state, and x_safety is safety state.

The controller then solves:

u*=argmax_u E[Utility(T_j,S(t+Δt))|S(t),u,model]−SafetyPenalty(u)−SleepFragmentationPenalty (u)

where u includes stimulation montage, amplitude, waveform, frequency, phase, timing, pulse structure, duty cycle, and duration.

After stimulation, the system measures whether the desired state was approached:

TE=f(S_observed_post−S_predicted_pre,T_j,sham/control,uncertainty)

The stimulation policy is then updated.

### Representative Stimulation Species

### Species 1—Slow-Oscillation N3 Enhancement

The system detects N2, high N2-to-N3 transition probability, or early N3. It delivers low-frequency tES, for example about 0.1-2 Hz or about 0.75 Hz, to enhance source-localized slow oscillation activity. Target engagement includes increased N3 probability, increased source-localized slow-wave activity, and decreased skin-path-corrected impedance or increased FAST-ECS. This is the current WISP species. The existing WISP filing already describes GC-window-gated stimulation, 0.1-2 Hz SO-tES, and GCI/RGCI optimization.

### Species 2—Respiratory-Phase CSF/ECS Enhancement

The system extracts respiratory phase and amplitude and times stimulation to a respiratory phase predicted to enhance CSF/ECS exchange. Stimulation may be delivered at a low-frequency carrier, as pulse trains, or as a phase-resetting perturbation. Target engagement includes increased respiratory phase-locking of FAST-ECS and improved respiratory-CSF coupling.

### Species 3—Cardiac/Perivascular Coupling Enhancement

The system extracts cardiac phase from ECG, PPG, EEG artifact, or EIT artifact. Stimulation is timed to pulse-driven perivascular dynamics or avoided during vulnerable cardiac/autonomic states. Target engagement includes increased pulse-locked FAST-ECS response and improved cardiac coupling.

### Species 4—Vasomotor/Infraslow Organization

The system estimates vasomotor oscillations in an infraslow band, for example about 0.01-0.20 Hz or about 0.05-0.15 Hz. Stimulation is delivered to enhance or stabilize infraslow neural-vascular-CSF organization. Target engagement includes increased coherence among source-localized infraslow activity, vasomotor signals, and FAST-ECS.

### Species 5—REM Stabilization or REM Clearance Enhancement

The system identifies REM or phasic REM, corrects for electrodermal/mastoid artifact, and delivers stimulation only when predicted to preserve or improve REM continuity without excessive arousal. Target engagement includes REM continuity, reduced arousal, and REM-associated FAST-ECS response. Recent research (Shofner et al., 2026) found REM impedance effects but also emphasized mastoid/skin electrodermal artifact and the need for high-density recordings to separate skin from intracranial impedance.

### Species 6—Arousal Suppression and Autonomic Calming

The system detects hyperarousal using beta activity, source-localized cortical arousal, heart-rate burden, HRV, EDA, respiration, and FAST-ECS suppression. Stimulation may be chosen to reduce arousal and sympathetic activation. Target engagement includes reduced beta/arousal burden, improved HRV, reduced EDA bursts, improved sleep continuity, and improved FAST-ECS.

### Species 7—Memory-Consolidation State

The system selects stimulation to promote sleep-dependent memory consolidation, using source-localized slow oscillations, spindle coupling, N3/REM organization, and optional next-morning cognitive/ERP outcomes. Target engagement includes SO-spindle coupling, N3/REM organization, and FAST-ECS improvement.

### Species 8—Attention/Readiness or Wake-State Modulation

During waking, the system estimates source-localized attention, fatigue, or readiness state and selects tES to move the subject toward a desired cognitive state while monitoring FAST-ECS, autonomic state, and safety. Target engagement may include EEG source pattern change, reaction-time or ERP change, reduced drowsiness, or improved cognitive task performance.

### Species 9—Affective/Autonomic Regulation

The system selects stimulation to move the subject toward a desired affective/autonomic state such as calm, reduced anxiety, reduced rumination, reduced emotional hyperarousal, or positive affect. The target is represented by measurable EEG source patterns, HRV, respiration, EDA, sleep quality, and optionally self-report.

### Species 10—Nonresponder Detection and Alternative Policy Selection

If a stimulation protocol changes EEG sources but does not change FAST-ECS, the system identifies preserved neural target engagement with impaired hydraulic target engagement. If neither EEG nor FAST-ECS changes, the system identifies stimulation nonresponse and may select an alternate frequency, montage, phase, respiratory timing, lower amplitude, or non-electrical intervention.

1. A system for estimating sleep-dependent intracranial fluid transport in a human subject, comprising: (a) an electrode array configured to acquire electroencephalographic signals and boundary impedance measurements from the subject during sleep; (b) a memory storing a probabilistic cerebrospinal-fluid-flow prior atlas comprising anatomical probabilities for cerebrospinal-fluid spaces, perivascular routes, parenchymal extracellular-space compartments, and outflow compartments; and (c) one or more processors configured to: (i) register the probabilistic cerebrospinal-fluid-flow prior atlas to a subject-specific anatomical model or to an atlas-scaled anatomical model; (ii) extract physiological driver signals comprising sleep stage and at least one of cardiac pulsatility, respiration, vasomotor oscillation, slow-oscillation activity, REM phasic activity, arousal, posture, or stimulation timing; (iii) compute an electrical forward model mapping regional conductivity or extracellular-space changes to the boundary impedance measurements; (iv) estimate, by Bayesian inference, a posterior state of one or more of cerebrospinal-fluid flow, perivascular flow, extracellular-space expansion, parenchymal exchange, or glymphatic-clearance capacity; and (v) output a map, score, stage, trajectory, or target-engagement metric representing the posterior state.

2. The system of claim 1, wherein the subject-specific anatomical model is derived from a T1-weighted magnetic resonance image of the subject, and wherein the atlas-scaled anatomical model is generated from one or more of head circumference, cranial landmarks, electrode positions, age, sex, anthropometrics, or impedance-transfer measurements.

3. The system of claim 1, wherein the probabilistic cerebrospinal-fluid-flow prior atlas comprises one or more of ventricles, cerebral aqueduct, basal cisterns, cisterna magna, subarachnoid space, foramen magnum, upper cervical cerebrospinal-fluid space, arterial territories, venous territories, venous sinuses, perivascular route probabilities, or driver-specific gain or lag maps.

4. The system of claim 1, wherein the boundary impedance measurements comprise FAST-EIT, FAST-ECS, single-frequency impedance, multi-frequency electrical impedance spectroscopy, transfer impedance, or electrical impedance tomography, and wherein the electrode array comprises a high-density array or a reduced-channel wearable array calibrated from a high-density array.

5. The system of claim 1, wherein the one or more processors are configured to compute skin-path-corrected intracranial impedance by suppressing skin, scalp, electrode-contact, sweat, electrodermal, or motion contributions using one or more of adjacent electrode pairs, superficial-control pairs, electrodermal activity, spatial covariance, adaptive filtering, regression, model-based inversion, or Bayesian source separation.

6. The system of claim 1, wherein the posterior state comprises a spatial probability map at a spatial scale finer than a native impedance reconstruction resolution obtainable without the probabilistic cerebrospinal-fluid-flow prior atlas, and wherein the one or more processors compute a model-adequacy score by comparing predicted FAST-EIT or FAST-ECS dynamics with observed FAST-EIT or FAST-ECS dynamics.

7. The system of claim 1, wherein the one or more processors are configured to classify the subject into a clearance-reserve stage using FAST-ECS features and physiological driver-response features, and to update the clearance-reserve stage using one or more biomarker measurements comprising Aβ40, Aβ42, Aβ42/Aβ40 ratio, p-tau217, p-tau181, GFAP, NfL, α-synuclein, phosphorylated α-synuclein, inflammatory markers, vascular markers, or metabolic markers.

8. The system of claim 1, wherein the one or more processors are further configured to estimate cortical source activity from high-density electroencephalographic signals registered to the same anatomical model, estimate a time-lagged relationship between the cortical source activity and the posterior state, and output a cortico-hydraulic coupling map, score, matrix, stage, or report.

9. The system of claim 1, further comprising a transcranial electrical stimulation subsystem, wherein the one or more processors are configured to detect an N2-to-N3 transition probability or early N3 sleep and deliver or schedule slow-oscillation transcranial electrical stimulation at about 0.1 Hz to about 2 Hz to enhance source-localized slow oscillation activity, with target engagement comprising increased N3 probability or slow-wave activity and decreased skin-path-corrected intracranial impedance or increased FAST-ECS.

10. The system of claim 1, further comprising a transcranial electrical stimulation subsystem, wherein the one or more processors are configured to extract respiratory phase or amplitude and time stimulation to a respiratory phase predicted to enhance cerebrospinal-fluid or extracellular-space exchange, with target engagement comprising increased respiratory phase-locking of FAST-ECS or improved respiratory-CSF coupling.

11. The system of claim 1, further comprising a transcranial electrical stimulation subsystem, wherein the one or more processors are configured to extract cardiac phase from ECG, PPG, EEG artifact, EIT artifact, or another cardiac signal and time or withhold stimulation based on pulse-driven perivascular dynamics or cardiac-autonomic vulnerability, with target engagement comprising increased pulse-locked FAST-ECS response or improved cardiac-perivascular coupling.

12. The system of claim 1, further comprising a transcranial electrical stimulation subsystem, wherein the one or more processors are configured to estimate vasomotor oscillations in an infraslow band of about 0.01 Hz to about 0.20 Hz and deliver stimulation to enhance or stabilize infraslow neural-vascular-CSF organization, with target engagement comprising increased coherence among source-localized infraslow activity, vasomotor signals, and FAST-ECS.

13. The system of claim 1, further comprising a transcranial electrical stimulation subsystem, wherein the one or more processors are configured to identify REM sleep or phasic REM sleep, correct boundary impedance measurements for electrodermal, mastoid, skin, or motion artifacts, and deliver stimulation only when predicted to preserve or improve REM continuity without excessive arousal, with target engagement comprising REM continuity, reduced arousal, or REM-associated FAST-ECS response.

14. The system of claim 1, further comprising a transcranial electrical stimulation subsystem, wherein the one or more processors are configured to detect hyperarousal using beta activity, source-localized arousal activity, heart-rate burden, heart-rate variability, electrodermal activity, respiration, or FAST-ECS suppression and select stimulation to reduce arousal or sympathetic activation, with target engagement comprising reduced beta or arousal burden, improved heart-rate variability, reduced electrodermal bursts, improved sleep continuity, or improved FAST-ECS.

15. The system of claim 1, further comprising a transcranial electrical stimulation subsystem, wherein the one or more processors are configured to select stimulation to promote sleep-dependent memory consolidation based on source-localized slow oscillations, spindle coupling, N3 organization, REM organization, or next-morning cognitive or electrophysiological outcomes, with target engagement comprising improved slow-oscillation-spindle coupling, N3/REM organization, or FAST-ECS.

16. The system of claim 1, further comprising a transcranial electrical stimulation subsystem, wherein, during waking, the one or more processors are configured to estimate source-localized attention, fatigue, or readiness state and select stimulation to move the subject toward a desired cognitive state while monitoring FAST-ECS, autonomic state, and safety, with target engagement comprising EEG source-pattern change, reaction-time change, event-related-potential change, reduced drowsiness, or improved cognitive-task performance.

17. The system of claim 1, further comprising a transcranial electrical stimulation subsystem, wherein the one or more processors are configured to select stimulation to move the subject toward an affective or autonomic target state comprising calm, reduced anxiety, reduced rumination, reduced emotional hyperarousal, positive affect, or improved stress recovery, the target state being represented by EEG source features, heart-rate variability, respiration, electrodermal activity, impedance, sleep quality, or self-report.

18. The system of claim 1, further comprising a transcranial electrical stimulation subsystem, wherein the one or more processors are configured to identify preserved neural target engagement with impaired hydraulic target engagement when stimulation produces a predicted cortical source change without producing a predicted FAST-ECS or glymphatic-clearance response, and to select an alternate frequency, montage, phase, respiratory timing, amplitude, stimulation dose, or non-electrical intervention.

19. A method for estimating and adaptively modulating intracranial fluid transport in a human subject, comprising: acquiring electroencephalographic signals and boundary impedance measurements from the subject; registering a probabilistic cerebrospinal-fluid-flow prior atlas to a subject-specific anatomical model or atlas-scaled anatomical model; extracting physiological driver signals comprising sleep stage and at least one of cardiac pulsatility, respiration, vasomotor oscillation, slow-oscillation activity, REM phasic activity, arousal, posture, or stimulation timing; estimating, by Bayesian inference and an electrical forward model, a posterior state of cerebrospinal-fluid flow, perivascular flow, extracellular-space expansion, parenchymal exchange, or glymphatic-clearance capacity; selecting a target neurohydraulic state from a target-state library; predicting stimulation-response signatures for candidate transcranial electrical stimulation protocols; selecting a stimulation protocol predicted to move the subject toward the target neurohydraulic state while satisfying safety constraints; delivering or scheduling stimulation according to the selected stimulation protocol; measuring a post-stimulation neurohydraulic state using electroencephalography and boundary impedance; computing target engagement from a cortical source response and a FAST-ECS response; and updating the posterior state, target-state library, or stimulation policy based on the target engagement.

20. A non-transitory computer-readable medium storing instructions that, when executed by one or more processors, cause the one or more processors to acquire electroencephalographic and boundary impedance data from a human subject, register a probabilistic cerebrospinal-fluid-flow prior atlas to a subject-specific or atlas-scaled anatomical model, extract physiological driver signals, estimate a posterior state of cerebrospinal-fluid flow or extracellular-space dynamics using Bayesian inference and an electrical forward model, classify a clearance-reserve stage or neurohydraulic target state, predict stimulation-response signatures for candidate stimulation protocols, select a stimulation protocol subject to safety constraints, compute target engagement from post-stimulation electroencephalographic and FAST-ECS responses, and update a model or stimulation policy.
